(19) United States
(12) Patent Application Publication
(10) Pub. No.: US 2009/0259097 A1
(43) Pub. Date: Oct. 15, 2009

(54) Cannula visualization arrangement

(76) Inventor: Ronald J. Thompson, Fort Thomas, KY (US)

Correspondence Address: Donald N. Halgren, 35 Central Street, Manchester, MA, 01944, US

(21) Appl. No.: 12/082,235

(22) Filed: Apr. 9, 2008

(51) Int. Cl. A61B 1/04

(52) U.S. Cl. 600/109

(57) ABSTRACT

The device described is a minimally-invasive surgical cannula that houses a miniature camera initially positioned at the inferior aspect of the distal cannula. Once indwelling in the operative space, the distal cannula ring is rotated 180° via a longitudinal pivot to reposition the camera superiorly. Repositioned, the camera is at a distance of twice the OD (OD) of the cannula. The manipulable cannula camera allows both minimally invasive operative instrumentation and superior operative vision via a single operative cannula.

### BACKGROUND OF THE INVENTION

1. Field of the Invention

This invention relates to surgical devices and more particularly to cannula with an articulable operation field visualization system arranged therewith.

2. Prior Art Introduction

The prior art can be somewhat confusing to evaluate because of different names assigned to the same instruments. What is currently called a trocar is actually a trocar assembly of an outer tubular cannula which may include a sharp distally tipped obturator (trocar) to incise the abdominal wall. In use, the sharp distal tip of the trocar (obturator) is used to incise the abdominal fascia and parietal peritoneum and then is removed from the cannula. Removing the trocar leaves the hollow, tubular cannula indwelling through the full thickness of the abdominal wall from the ambient environment to the intra-abdominal operative environment.

U.S. Pat. No. 5,385,553, “Trocar with Floating Septum Seal,” describes an actual cannula seal, but refers to the seal as a “trocar seal”. U.S. Pat. Nos. 5,707,362 and 6,447,527 describe different sharp obturator instruments for the penetration of tissue. The current invention only relates to the distal aspect of the tubular cannula, not the proximal cannula where airtight seals are located, and not the various methods of incising the abdominal wall for placement of the cannula through the abdominal wall.

Prior Art

U.S. Pat. No. 6,524, 283, “Method and Apparatus for Anchoring Laparoscopic Instruments,” describes a balloon anchor on the distal cannula inserted through the abdominal wall deflated, and then inflated beyond the OD of the distal cannula for cannula stabilization after insertion through the abdominal wall. U.S. Pat. No. 6,056,766, “Stabilized Trocar and Method of Using Same,” describes an elliptical distal cannula element to accomplish cannula, not trocar, stabilization that prevents the cannula from being withdrawn from the abdominal wall during the withdrawal of surgical instruments through the cannula.

U.S. Pat. No. 5,166,787 describes an endoscope having a provision for repositioning a video sensor to a location which does not provide the same cross-sectionally viewed relationship with the distal end. U.S. Pat. No. 5,305,121 describes a “Stereoscopic Endoscopic System” that uses tandem or double-barreled laparoscopes. Most importantly, U.S. Pat. No. 5,895,350, “Electric Endoscope,” issued Apr. 20, 1999, describes a standard laparoscope with a window in the distal end, a lens proximate to the window, and a photodetector proximate to the lens. The electronic image is captured by the photodetector cell and hardwired through the hollow laparoscope to exit at the proximate end and attached to a video monitor. U.S. Pat. No. 7,322,934 describes a flexible gastroscope with a distal head unit freely detached from the distal inserting unit that includes an image pick-up device and a light-emitting element.

Capsule endoscopy technology (PILL CAM) is a self-contained light-emitting, photodetection camera and a miniaturized transmitter described in numerous US Patents, including U.S. Pat. Nos. 6,709, 387; 6,836,377; 6,904,308; 6,904,308; 6,934,093; 7,083,578; and 7,214,182. This capsule endoscopy technology is an alternative to the gastroscope for the visual internal examination of the upper gastrointestinal tract and the small bowel and replace the flexible gastroscope. The technology was developed by Given Imaging, LTD. The last patent cited above, U.S. Pat. No. 7,214,182, is entitled “Wireless In-Vivo Information Organizing System, Body-Insertable Device, and External Device.” It describes acquiring a real-time image inside the body and transmitting that image to a video monitor outside of the body for viewing on an external monitor.

### BACKGROUND

Endoscopes are medical devices that are useful for viewing and accomplishing specific procedures in body orifices or cavities. Endoscopes all have three important capabilities: the ability to transmit light for the illumination of the body orifice or cavity, the ability to transmit air or a gaseous fluid to distend and inflate the orifice or cavity, and the ability to allow vision at a distance within that orifice or cavity. The hollow channel (or channels) for transmitting air or a gaseous fluid to distend and inflate the orifice or cavity can also be used to introduce operative instruments, thus allowing specific medical/surgical procedures to be done at a distance from the physician or surgeon. While all endoscopes have distal and proximal ends, they do vary in length and can be either flexible or rigid. Further, each type of endoscope will have a specific name relative to the body orifice or cavity it is intended to be used in. A partial history of these endoscopes includes:

| Flexible Endoscopes: | Rigid Endoscopes: |
|---|---|
| 1. Bronchoscope | 1. Arthroscope |
| 2. Esophagoscope | 2. Cystoscope (Urinary Bladder) |
| 3. Gastroscope | 3. Laryngoscope |
| 4. Colonoscope | 4. Hysteroscope (Uterine Cavity) |
| | 5. Laparoscope (Abdominal Cavity) |

### Recent History of Abdominal Surgery/Gynecologic Surgery

The laparoscope has become the standard for most general surgical procedures since the early 1990's. In 2006, eight hundred thousand laparoscopic cholecystectomies (gall bladder removals) were completed in the US alone. Laparoscopic abdominal and gynecologic surgeries are also referred to as minimally invasive surgeries. These are termed ‘minimally invasive’ because they utilize the insertion of instruments via multiple small incisions to accomplish the surgical procedure, in contrast to more traditional routes which require a ten inch incision into the abdominal cavity (a laparotomy).

Gynecologists began using laparoscopes in the 1960's and 1970's for simple minimally invasive gynecologic procedures, such as diagnosis of painful pathologic gynecologic conditions such as endometriosis and for simple operative procedures such as tubal occlusive procedures for sterilization. Diagnostic exams utilize a diagnostic laparoscope without any operative channel. The initial operative tubal occlusive procedure was the tubal fulgeration where bipolar electrical (RF) energy was used to dessicate the fallopian tubes and cause the tubal lumens to scar closed. Subsequent improved tubal occlusion methodology included the falope ring and the Hulka clip in the 1980's. All three methods of tubal occlusion were accomplished through the 8 mm OD operating channel of the rigid operative laparoscope.

In the mid 1980's gynecologists expanded operative laparoscope procedures to laser (CO2) lysis of adhesions, laparoscopic removal of tubal pregnancies, laparoscopic ovarian cystectomies, and laparoscopic salpingoophorectomies (removal of the fallopian tube and ovary). These increasingly sophisticated operative laparoscopic surgeries were accomplished through peripherally placed trocar/cannulas, not through the operative channel of the laparoscope. Specific laparoscopic instrumentation included tissue graspers, scissors, surgical clip applicators, and linear stapler/cutters allowed the progressive advancement of minimally invasive gynecologic surgery. With this advancement to more complex laparoscopic procedures the operative laparoscopic instrument was abandoned and replaced by an improved panoramic diagnostic laparoscope. By the late 1980's almost all operative laparoscopic gynecologic procedures used a 10 mm umbilically placed panoramic laparoscope for light and vision only, and accomplished surgical procedures with laparoscopic instruments through three or four peripherally placed cannulas.

Vision during surgery utilizing the panoramic laparoscope is accomplished with a camera that is affixed to the proximal end, otherwise known as the eyepiece. This camera is hard wired to multiple television monitors positioned on either side of the anesthesized patient. Further, these monitors are stationed on pivoting mounts allowing their adjustment at or above the eye level of the operating surgeon. This arrangement of the television monitors is quite awkward relative to the surgical instrumentation used to accomplish the procedure. Nonetheless, seventeen years later this awkward arrangement is still the standard.

In the late 1980's two physicians (a gynecologic surgeon specializing in technically advanced laparoscopic surgeries and a general surgeon) paired up to develop a laparoscopic procedure to remove the gall bladder. The laparoscopic cholecystectomy was initially taught to general surgeons by skilled gynecological laparoscopic surgeons. This laparoscopic cholecystectomy was rapidly adopted by general surgeons due to the practicality of minimally invasive surgery, and by the mid 1990's general surgeons became adept at removing the gall bladder laparoscopically. Since the mid 1990's general surgeons have become very skilled with operative laparoscopic procedures, including bowel resections, appendectomies, and other complicated surgical procedures. General surgeons now accomplish eighty percent of all operative laparoscopic procedures, with gynecologic surgeons accomplishing the other twenty percent. General surgeons have always used the panoramic diagnostic laparoscope and have never even considered using the umbilical site as an operative port.

### Limited Vision Problems with Operative Laparoscopes

The OD of the operative laparoscope with an 8 mm operating channel is 12 mm. The OD of the operative laparoscope with a 5 mm operating channel is 10 mm. The vision fiber optic bundle within the operative laparoscope is positioned superior to the operating channel of the laparoscope. When an instrument is placed through the operative channel of the laparoscope and advanced into the operative space, all inferior vision becomes obstructed by the instrument, reducing vision by about forty percent of the operative field. With “simple” operative procedures such as a tubal occlusion, this loss of total vision is acceptable, however with more complicated procedures this loss of vision can be unacceptable.

### BRIEF SUMMARY OF THE PRESENT INVENTION

The present invention relates to a cannula that provides all of the functions of an operative laparoscope: light for illumination of the operative field, an operative channel allowing for cavity inflation and instrumentation entry, and the ability to allow vision of the operative field. The vision of this inventive cannula will be relatively unobstructed by the operating channel instrumentation due to positioning the distal vision camera outside of the OD of the cannula. The position of the camera will be not only outside of the cannula, but at a distance of 12-30 mm above the OD of the cannula to provide superior vision of the entire laparoscopic operative field. This is accomplished by transecting the distal end of the cannula (distal cannula ring), and fixing the distal cannula ring to the distal body of the cannula with a pivot located at the superior aspect of the cannula. By positioning the camera in the inferior aspect of the cannula ring and rotating the entire cannula ring by at least a 180° re-positioning within the body cavity, the camera will be re-positioned superior to the cannula and operative channel. This re-positioning would be equal to the ID of the cannula. If the cannula ID is 12 mm, the camera will be 10-12 mm above the superior periphery of the cannula, or 18 mm above the center of the cannula. If the ID of the cannula is 20 mm, the vision lens or cameras will be 18-20 mm above the superior periphery of the cannula, or 10 mm above the center of the cannula. Therefore, the 180° rotation of the distal camera support member, ie. cannula ring, outside of the confines of the cannula periphery allows a superior view of the operative field from the camera contained in the distal cannula ring. The 180° rotation is afforded by a rotational shaft that is fixed to the distal cannula ring, courses the entire length of the cannula, and is hand manipulated at the proximal end of the cannula located outside of the patient's abdominal cavity.

In operative functioning, the operative cannula would initially be fitted with the introducing trocar. In this configuration, the distal camera supporting cannula ring would be aligned with the operative cannula. Once placed into the pneumoperitoneum, the trocar component would be withdrawn, and the proximal rotational shaft actuator rotated 180° causing the distal cannula ring to likewise rotate 180°. This would rotate the camera from an inferior initial position to an operative superior position. This would lock in place externally for the entire operative procedure. Once the operative procedure is completed, the proximally located rotational shaft actuator would be unlocked, rotated back to the insertion position, and withdrawn from the operative pneumoperitoneum. For withdrawal, the distal cannula ring would be, as on insertion, continuous circumferential alignment with the operative cannula.

The two hallmark features of the present cannula invention are: 1) 180° rotational transformation of the camera-containing distal cannula ring once the cannula is inside the operative pneumoperitoneum; and 2) an entirely unobstructed operative field even with the introduction of instruments through the operative cannula. The end result of these two features is improved endoscopic vision. The benefit of the present cannula invention is that it enables the re-introduction of an operative port, as well as improved operative visualization at the umbilical incision site or any other cannula site.

The shaft of the cannula may in a further embodiment, have longitudinal ridges in-line with the axis of the cannula to prevent rotation of the cannula during operative instrumentation. Multiple currently-marketed cannulas have circular ridges on the OD of the cannula, perpendicular to the axis of the cannula, to prevent withdrawal of the cannula from the abdominal wall during operative instrumentation. The longitudinal ridges of the inventive cannula would interface with the rectus fascial incision, not to prevent cannula withdrawal, but to prevent rotation of the cannula relative to the fascial incision. The longitudinal ridges are intended to maintain proper visual orientation with the distal camera always in a superior position relative to the shaft of the cannula.

The invention thus comprises a cannula arrangement for operating upon and permitting the unobstructed viewing of an operative field within a patient being operated upon, comprising: an elongated hollow cannula body having a proximal end and a distal end; and a camera mounted on an articulable member attached to the distal end of a manipulable handle extending longitudinally through a wall of the cannula body, the camera arranged in electronic communication with a communications link through the cannula body. The articulable member preferably comprises a camera supporting cannula ring. The cannula ring is preferably arranged to pivot about a pivot axis on the distal end of the cannula. The cannula ring preferably has illumination members thereon to permit lighting of the operative field within a patient. The manipulable handle may have in a further embodiment, a proximal end consisting of a generally “C” shaped curve for permitting movably securable engagement with a securement location on the cannula. The securement location on said cannula preferably comprises at least one transversely directed channel in the outer wall of the cannula arranged to receive the “C” shaped curve, once the cannula ring has been pivoted from an “in-line” orientation to a ring-superior orientation with respect to the cannula.

The invention also comprises a method of utilizing a cannula for operating upon and permitting the unobstructed viewing of an operative field within a patient being operated upon, comprising one or more of the following steps, including: attaching a camera on an articulable member attached to the distal end of a manipulable handle extending longitudinally through a wall of said cannula body, the camera arranged in electronic communication through a network to a monitor outside of the patient; moving the articulable camera ring member supporting the camera into a cannula superior orientation by moving the manipulable handle, once the distal end of the cannula has been introduced into the operative field of the patient; and securing the proximal end of the manipulable handle into a channel arranged on the outer side of the cannula to lock the camera in the cannula-superior orientation. The method included rotation of the manipulable handle about its longitudinal axis to move the camera from a cannula inferior orientation about a pivot axis, through an arc of about 180 degrees, to a secured, cannula superior orientation. Rotation of the manipulable handle about its longitudinal axis is effective to move an illumination arrangement to a secured cannula superior orientation from a cannula inferior orientation. The manipulable handle may in a further embodiment, be moved axially/longitudinally to permit the annular ring camera support member to be adjustably supported/extended beyond the distalmost end of the body of the cannula. The camera support ring may be of generally semi-circular shape in a farther embodiment thereof. The method may include arranging a plurality of parallel, longitudinally aligned, patient-tissue-engaging ridges on the surface of the cannula to stabilize the cannula and minimize any rotation of the cannula with respect to the patient during any manipulation of the handle.

The invention also may include securing the proximal end of the manipulable handle into a second channel arranged adjacent the first channel on the outer side of the cannula to lock the camera in a second, distally-advanced, cannula-superior orientation. The electronic communications network may comprise a fiber optic link extending through the control shaft to the monitor arranged outside of the patient.

The invention may also comprise a method of utilizing a cannula assembly for operating upon and permitting the unobstructed viewing by a monitor, of an internal operative field of a patient being operated upon, comprising: moving controllably, a camera and illumination patient-internal-operative-site viewing and lighting arrangement from a cannula “in-alignment” orientation, to a cannula “superior” orientation upon advancement of the cannula assembly within the patient's operative site; articulating the viewing and lighting arrangement by a cannula proximal handle and a co-extensive control shaft member supported by the cannula; communicating viewing images through the shaft to the monitor, wherein the articulation comprises both radial and axial movement of the camera.

### BRIEF DESCRIPTION OF THE DRAWINGS

The objects and advantages of the present invention will become more apparent when viewed in conjunction with the following drawings in which:

FIG. 1 is an end view of the distal end of an operative laparoscope (cannula) of the prior art;

FIG. 1A is a representation of the field of vision of the prior art cannula represented in FIG. 1;

FIG. 2 is an end view of the distal end of an operative laparoscope (cannula) of a 10 mm panoramic operative laparoscope;

FIG. 2A is a representation of the field of vision of the cannula represented in FIG. 2;

FIG. 3 is a perspective view of the cannula constructed according to the principles of the present invention;

FIG. 4 is a perspective view of the cannula shown in FIG. 3, with the distal cannula ring in a rotated orientation;

FIG. 4A is a distal end view of the cannula and rotated cannula ring shown in FIG. 4;

FIG. 4B is a representation of the field of vision of the inventive cannula shown in FIG. 3;

FIG. 5 is a perspective view of the inventive cannula in an operative configuration within an abdomen of a patient;

FIG. 6 is a side elevational view of the cannula with a further embodiment of the manipulable handle; and

FIG. 7 is an end view of a further embodiment of the camera enclosing articulable member.

### DETAILED DESCRIPTION OF THE PRESENT INVENTION

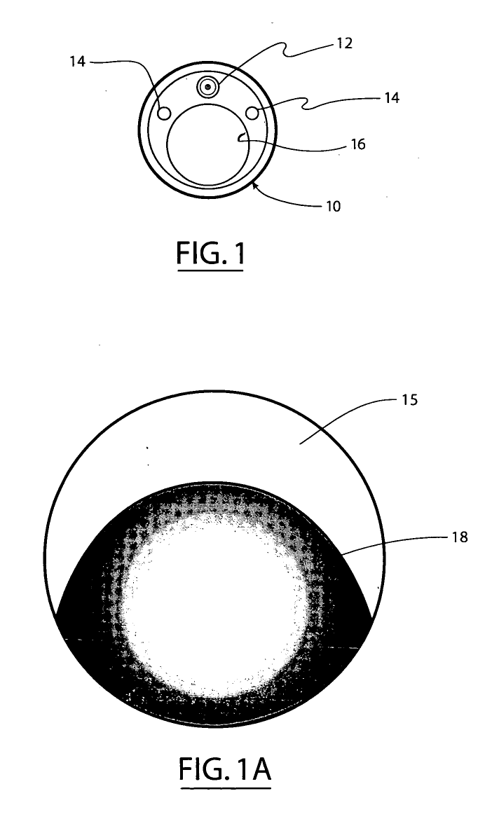

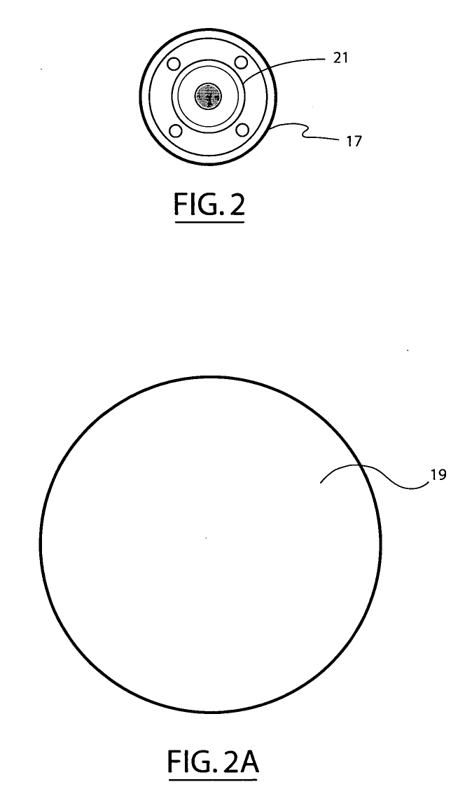

Referring now to the drawings in detail, and particularly to FIG. 1 there is shown an example of the distal end of a “prior art” 12 mm operative laparoscope (cannula) 10 showing a fiber optic vision channel 12, a set of fiber optic illumination means 14 and an operative channel 16 through which surgical instruments, not shown, are utilized. FIG. 1A represents the limited “crescent-shaped” limited field of vision 15 of such a prior art scope 10, with an operative instrument 18 being utilized within that channel 16. FIG. 2 represents a distal end view of a 10 mm panoramic laparoscope 17. FIG. 2A represents a view 19 through a fiber optic channel 21 which extends through a laparoscope/cannula 17, which view 21 however would be severely limited during a procedure.

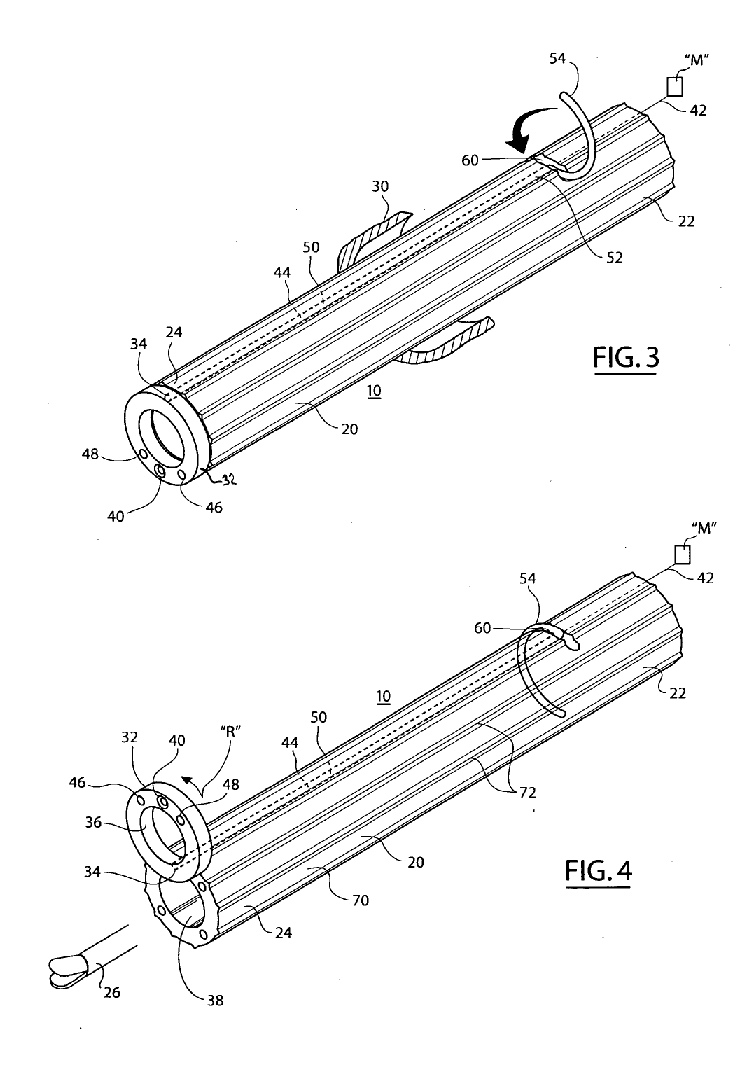

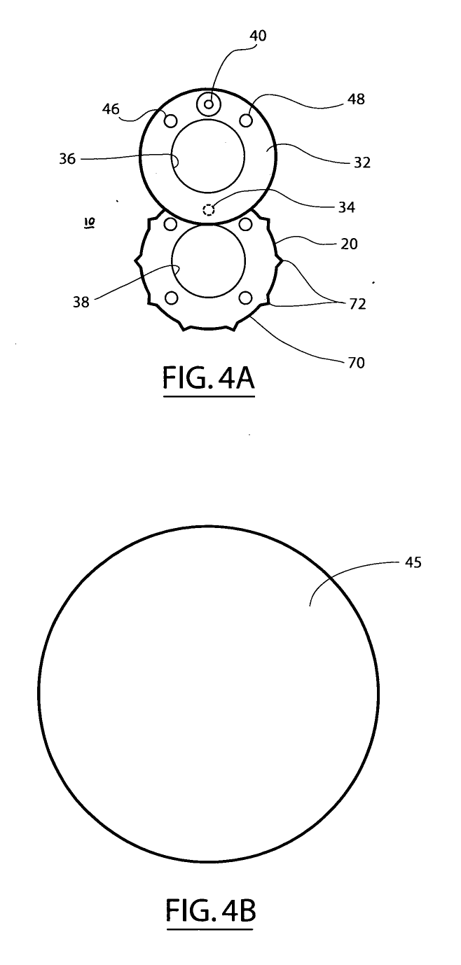

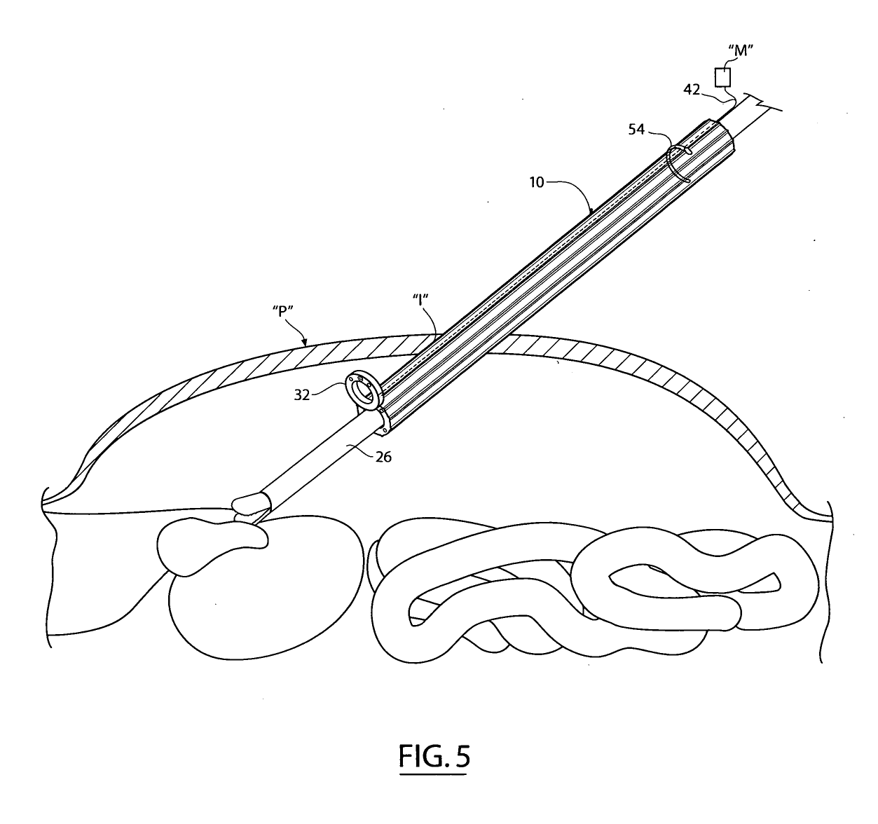

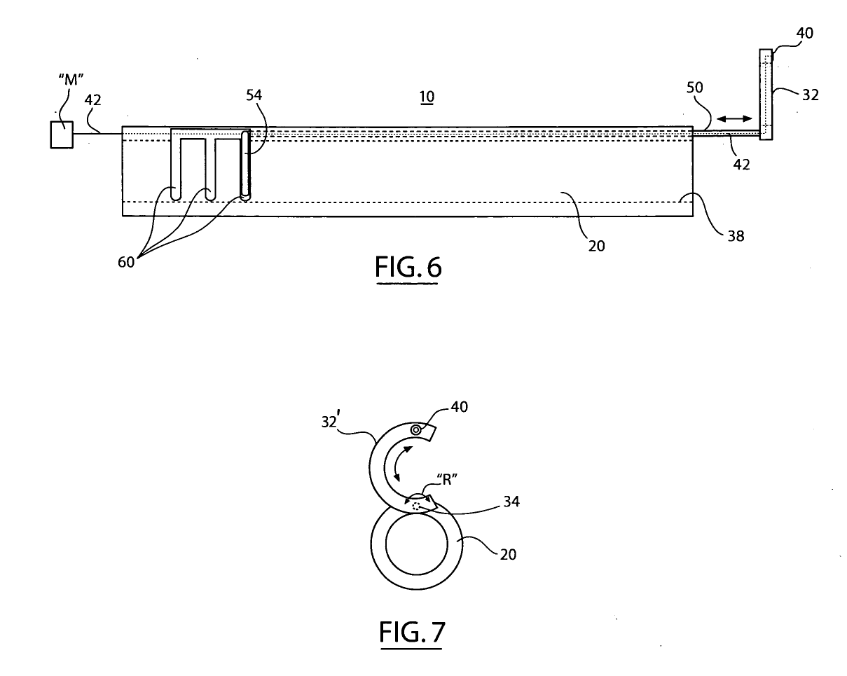

The cannula 10 of the present invention, as represented in FIGS. 3 and 4 comprises an elongated generally cylindrically shaped tubing 20 having a proximal end 22 and a distal end 24, which distal end 24 would be introduced into a patient “P” through an introductory trocar 30, shown only partially in FIG. 3, and also shown in FIG. 5. Both of the ends 22 and 24 of the cannula 10 are open for the receipt/passage of an operative surgical instrument(s) 26, therethrough. The distal end 24 of the cannula 10 has an articulable camera support member 32 such as for example, distal cannula ring mounted thereon, so as to be moved relative to the distal end 24 of the cannula 10, for example, a pivot movement about a pivot axis 34 arranged at the distalmost end 24 of the cannula 10.

In one preferred embodiment, the distal cannula ring 32, has a central opening 36 therethrough which is in communicative alignment with the operating channel 38 of the cannula 10, as represented in FIG. 3. The distal cannula ring 32 has a miniaturized camera 40 arranged at a location diametrically opposed to the pivot axis 34 of that distal cannula ring 32. The camera 40 is in communication with a monitor “M”, through a flexible fiber optic conduit 42 or the like, extending within the ring 32 and through both the pivot axis 34 and a channel 44 within the sidewall of the cannula 10. Further preferred embodiments of the camera 40 arrangement may consist of an rf communicative camera 40 within that support member 32, which is in wireless communication with the monitor “M”.

A pair of fiber optic illumination ports 46 and 48 are disposed adjacent the camera 40 at the distal edge of the distal cannula ring 32. The fiber optic lines 42 embodiment extend through the distal cannula ring 32 and through an opening at the pivot axis 34, proximally through the wall of the cannula tube 20, to the monitor “M”, to comprise a control and monitoring arrangement connected with the cannula assembly 10.

An elongated rigid rotation shaft 50, which is preferably hollow, for enclosure of the longitudinally extending optic lines 42, is attached to the rear side of the distal cannula ring 32, at its pivot axis 34. The elongated rigid rotation shaft 50 extends linearly along and through the wall of the cannula body having a proximalmost end 52 generally near the proximal end 22 of the cannula 10. A further embodiment comprises such rotation shaft 50 externally attached very closely to the cylindrical body of the cannula 10, not shown in the figures for clarity of views. A generally “C”-shaped rotation shaft handle 54 is rigidly attached to the proximalmost end 52 of the rotation shaft 50, as represented in FIG. 3. The rotation shaft handle 54 is pivotable about the longitudinal axis of the rotation shaft 50, so as to effect rotation of that rotation shaft 50 as well as rotation of the distal, articulable camera support member, ie. the cannula ring 32, about its pivot axis 34 at the distal end 24 of the cannula 10.

At least one generally semi-circumferential channel 60 is formed into the body of the cannula, adjacent its proximalmost end 22, so as to receive the generally “C”-shaped rotational shaft handle 54, as represented for example, in FIG. 4, once that handle 54 is rotated about its longitudinal axis of the shaft 50, so as to enclose and snappingly lock the distal cannula ring 32 pivoted thereby, into a locked orientation of a superior aspect for the camera 40. That is, the distal cannula ring 32 is rotated about 180 degrees about its pivot axis 34, as represented by arrow “R”, to effect and improve the visualization field of that cannula system 10 by moving the camera 40 and lights 46 and 48 out of the way of any instrumentation and into a cannula “superior” orientation.

During a surgical procedure, the cannula 10 is inserted into an abdominal cavity of a patient, through an introducing trocar 30, as is represented in FIG. 1, which trocar 30 may be subsequently removed after the cannula 10 is positioned within a patient, as represented in FIG. 5. The exterior wall surface 70 of the cannula 10, in one preferred embodiment thereof, have a plurality of parallel, longitudinally arranged anti-rotation ridges 72 extending therealong, as represented in the end view shown in FIG. 4A, and as also represented in FIGS. 3 and 4. Those ridges 72 act to minimize the likelihood of any rotation of the body of the cannula 10 within the operative incision “I” of a patient “P”, to prevent the rotation of that cannula 10 relative to the fascial incision “I”. Operative instruments 26 would of course be utilized through the bore 38 of the cannula 10 and out through its distal end 24, preferably after that distal cannula ring 32 has been rotated 180 degrees about its pivot axis 34 so as to maximally displace the camera 40 and thereby provide maximum operative field of vision 45, as represented in FIG. 4B. Such rotation of the camera support member or ring 32 may be accomplished after operative instruments are extended through the cannula 10 if such member 32′ is of generally semi-circular or generally “C” shape, as represented in the end view shown in FIG. 7. Distal and radial extended movement of the camera 40 may also be accomplished in a further embodiment wherein said shaft 50 and camera support is comprised of a shape memory metal such as for example Nitinol, wherein a camera support 32 flexes radially away from alignment with the bore or surgical instrument passageway of the cannula 10, as that camera support is moved distally relative to the distal end 24 of the cannula 10.

1. A cannula arrangement for operating upon and permitting the unobstructed viewing of an operative field within a patient being operated upon, comprising:
an elongated hollow cannula body having a proximal end and a distal end; and
a camera mounted on an articulable camera support member attached to the distal end of a control shaft extending longitudinally through a wall of said cannula body, with a manipulable control handle proximally located thereon, the camera being arrangeable in electronic communication through a communications link within the cannula arrangement, to an external, operative-site monitor.

2. The cannula arrangement as recited in claim 1, wherein said articulable camera support member comprises a cannula ring.

3. The cannula arrangement as recited in claim 1, wherein said camera support member is arranged to pivot about a pivot axis of the control shaft, extending out of the distal end of the cannula.

4. The cannula arrangement as recited in claim 3, wherein said camera support member has at least one illumination member thereon to permit articulable lighting of the operative field within a patient.

5. The cannula arrangement as recited in claim 1, wherein said manipulable control handle has a proximal end consisting generally of a “C” shaped curve for movable securable engagement of the handle within a securable handle-receiving location on the cannula.

6. The cannula arrangement as recited in claim 5, wherein said securable handle-receiving location on said cannula comprises a transversely directed first channel on an outer wall of the cannula, the channel arranged to receive the “C” shaped curved manipulable handle once said camera support member has been moved from a cannula “in-line” orientation to a cannula superior orientation.

7. The cannula arrangement as recited in claim 1, wherein the cannula body has an exterior surface with a plurality of generally parallel patient-tissue-engaging ridges extending longitudinally thereon, to minimize rotative movement of the cannula during an operative procedure.

8. The cannula arrangement as recited in claim 6, wherein said securable handle-receiving location comprises a second channel on the cannula, parallel to and spaced longitudinally adjacent said first channel to permit longitudinal adjustment of the camera therewith.

9. The cannula arrangement as recited in claim 4, wherein the camera support member has a plurality of illumination members spaced thereon to permit multiple light displays upon the surgical site within the patient.

10. A method of utilizing a cannula for operating upon and permitting the unobstructed viewing of an operative field within a patient being operated upon, comprising:
arranging a camera on an articulable camera support member directed at the distal end of a control shaft connected to a manipulable handle, the camera arranged in electronic communication through an electronic communications network to a monitor outside of the patient;
moving the articulable camera support member into a cannula superior orientation by moving the manipulable handle, once the distal end of the cannula has been introduced into the operative field of the patient.

11. The method as recited in claim 10, including:
securing the proximal end of the manipulable handle into a first channel arranged on the outer side of the cannula to lock the camera in the cannula-superior orientation.

12. The method as recited in claim 10, wherein rotation of the manipulable handle about its longitudinal axis moves the camera from a cannula inferior orientation about a pivot axis, through an arc of about 180 degrees, to a secured, cannula superior orientation.

13. The method as recited in claim 10, wherein rotation of the manipulable handle about its longitudinal axis moves an illumination arrangement to a secured cannula superior orientation from a cannula inferior orientation.

14. The method as recited in claim 10, including:
arranging a plurality of parallel patient-tissue-engaging ridges on the surface of the cannula to stabilize the cannula and minimize any rotation of the cannula with respect to the patient during any manipulation of the handle.

15. The method as recited in claim 11, including
securing the proximal end of the manipulable handle into a second channel arranged adjacent the first channel on the outer side of the cannula to lock the camera in a second, distally-advanced, cannula-superior orientation.

16. The method as recited in claim 12, wherein the electronic communications network comprises a fiber optic link extending through the control shaft to the monitor arranged outside of the patient.

17. A method of utilizing a cannula assembly for operating upon and permitting the unobstructed viewing by a monitor, of an internal operative field of a patient being operated upon, comprising:
moving controllably, a camera and illumination patient-internal-operative-site viewing and lighting arrangement from a cannula “in-alignment” orientation, to a cannula “superior” orientation upon advancement of the cannula assembly within the patient's operative site.

18. The method as recited in claim 17, including:
articulating the viewing and lighting arrangement by a cannula proximal handle and a co-extensive control shaft member supported by the cannula.

19. The method as recited in claim 18, including:
communicating viewing images through the shaft to the monitor.

20. The method as recited in claim 19, wherein said articulating comprises both radial and axial movement of the camera.
